(19) United States
(12) Patent Application Publication
(10) Pub. No.: US 2025/0064702 A1
(43) Pub. Date: Feb. 27, 2025

(54) OIL-IN-WATER EMULSION COSMETIC

(71) Applicant: SHISEIDO COMPANY, LTD., Tokyo (JP)

(72) Inventors: Seiji NISHIKAWA, Tokyo (JP); Ikuhiro SUZUKI, Tokyo (JP); Yusuke OKUYAMA, Tokyo (JP)

(73) Assignee: SHISEIDO COMPANY, LTD., Tokyo (JP)

(21) Appl. No.: 18/711,955

(22) PCT Filed: Dec. 8, 2022
(86) PCT No.: PCT/JP2022/045227

§ 371 Date: May 21, 2024

(30) Foreign Application Priority Data
Dec. 21, 2021 (JP) 2021-207293

(51) Int. Cl. A61K 8/49; A61K 8/06; A61K 8/36; A61Q 19/08

(52) U.S. Cl. CPC A61K 8/4946; A61K 8/062; A61K 8/361; A61Q 19/08

(57) ABSTRACT

[Problem] To provide an oil-in-water emulsion cosmetic having an excellent feel in use.

[Solution] An oil-in-water emulsion cosmetic comprising (A) a cyclic carboxamide derivative having a specific structure or its salt, (B) a straight-chain higher fatty acid having 16 or more carbon atoms, (C) a higher alcohol, (D) a nonionic surfactant, (E) an oil, and (F) water.

### TECHNICAL FIELD

The present invention relates to an oil-in-water emulsion cosmetic.

### BACKGROUND ART

As an emulsification technique for generating fine emulsified particles, a liquid crystal emulsification method is known. The use of a liquid crystal phase for emulsification is considered to enable reduction of interfacial tension and formation of a high-strength interfacial film. For example, an oil-in-water emulsified composition containing a higher fatty acid, a higher alcohol, and a nonionic surfactant and having excellent emulsion stability has been proposed (Patent Literature 1). The users of such a composition have desired a more excellent feel in use, and there has been room for further improvement in the feel in use.

Meanwhile, a cyclic carboxamide derivative is known to have an anti-wrinkle effect and a pigmentation inhibitory effect, and is proposed to be blended in a cosmetic and the like (Patent Literature 2).

### CITATION LIST

### Patent Literature

Patent Literature 1: WO 2021/125051 A
Patent Literature 2: WO 2011/040496 A

### SUMMARY OF THE INVENTION

According to the studies of the present inventors, it has been found that use of a cyclic carboxamide derivative in a cosmetic tends to cause a sticky feel. The present inventors have surprisingly found that an excellent feel in use can be achieved by using an oil-in-water cosmetic comprising a combination of a higher fatty acid, a higher alcohol, a nonionic surfactant, and a specific cyclic carboxamide derivative. The present invention is based on these findings.

According to the present invention, the following invention is provided.
[1] An oil-in-water emulsion cosmetic comprising:
   (A) a cyclic carboxamide derivative or a salt of the cyclic carboxamide derivative, the cyclic carboxamide derivative represented by Formula (1):

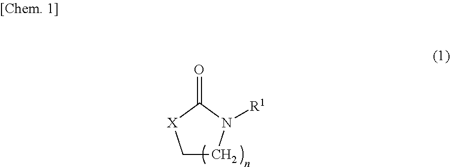

wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) a straight-chain higher fatty acid having 16 or more carbon atoms;
(C) a higher alcohol;
(D) a nonionic surfactant;
(E) an oil; and
(F) water.
[2] The cosmetic according to [1], wherein
   in Formula (1) of the component (A),
   R¹ represents a hydroxyalkyl group having 1 to 3 carbon atoms,
   X represents —CH₂— or —NH—, and
   n is 1.
[3] The cosmetic according to [1] or [2], wherein the component (A) is 1-(2-hydroxyethyl)-2-imidazolidinone.
[4] The cosmetic according to any one of [1] to [3], wherein an amount of the component (A) blended is 1 to 5 mass % with respect to a total amount of the cosmetic.
[5] The cosmetic according to any one of [1] to [4], wherein the component (B) is selected from the group consisting of behenic acid and stearic acid.
[6] The cosmetic according to any one of [1] to [5], wherein an amount of the component (B) blended is 0.1 to 3 mass % with respect to the total amount of the cosmetic.
[7] The cosmetic according to any one of [1] to [6], wherein the component (C) is selected from the group consisting of cetyl alcohol, isostearyl alcohol, stearyl alcohol, and behenyl alcohol.
[8] The cosmetic according to any one of [1] to [7], wherein an amount of the component (C) blended is 0.1 to 5 mass % with respect to the total amount of the cosmetic.
[9] The cosmetic according to any one of [1] to [8], wherein the component (D) is selected from the group consisting of polyoxyalkylene glycol fatty acid esters and polyether-modified silicones.
[10] The cosmetic according to any one of [1] to [9], further comprising (G) an alkyl glyceryl ether.
[11] The cosmetic according to any one of [1] to [10], further comprising (H) nicotinic acid or a derivative of nicotinic acid.

According to the present invention, an oil-in-water emulsion cosmetic having an excellent feel in use can be provided. In particular, a sticky feel is suppressed after application, and a moist feel and a smooth feel can be imparted to the skin. Furthermore, a mellow feel of a base can be felt.

### BEST MODE FOR CARRYING OUT THE INVENTION

The present invention relates to an oil-in-water emulsion cosmetic (hereinafter, sometimes referred to as a cosmetic) comprising (A) a cyclic carboxamide derivative having a specific structure or its salt, (B) a straight-chain higher fatty acid having 16 or more carbon atoms, (C) a higher alcohol, (D) a nonionic surfactant, (E) an oil, and (F) water.

In the cosmetic, emulsified particles (oil droplets) are dispersed in an aqueous phase. It is considered that the emulsified particles maintain the emulsion stability by a structure in which an interfacial film such as a gel film or a liquid crystal film based on a lamellar liquid crystal or the like, which is stronger than a micellar structure or the like, encloses an oil.

The average particle size of the emulsified particles is not particularly limited, but is preferably 20 μm or less, more preferably 10 μm or less, still more preferably 5 μm or less, and still even more preferably 3 μm or less immediately after preparation. The lower limit of the average particle size is preferably 500 nm or more, more preferably 700 nm or more, and still more preferably 1 μm or more. The average particle size can be specified as the average of the projected area circle equivalent diameters of 10 or more, preferably 100 or more emulsified particles observed with an optical microscope.

The viscosity of the cosmetic according to the present invention is not particularly limited, but is preferably 1,000 to 100,000 mPa·s, more preferably 5,000 to 50,000 mPa·s, and particularly preferably 10,000 to 30,000 mPa·s. The viscosity can be measured at 30° C. using a BH type viscometer.

### (A) Cyclic Carboxamide Derivative or its Salt

The cosmetic according to the present invention comprises (A) a cyclic carboxamide derivative represented by Formula (1) or its salt (hereinafter, sometimes referred to as a component (A), and the same applies to other components).

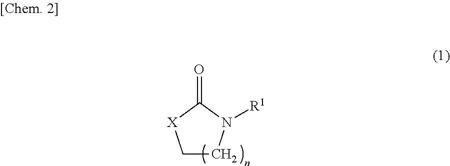

In Formula (1),
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3

The hydrocarbon group is not particularly limited, and may be, for example, an alkyl group, a cycloalkyl group, an alkenyl group, an alkynyl group, a cycloalkylalkyl group, a haloalkyl group, an alkoxyalkyl group, or an alkoxycarbonylalkyl group, and is preferably an alkyl group.

In a preferred embodiment, in Formula (1) of the component (A),
R¹ represents a hydroxyalkyl group having 1 to 3 carbon atoms,
X represents —CH₂— or —NH—, and
n is 1.

Specific examples of the cyclic carboxamide derivative represented by Formula (1) comprise the following.

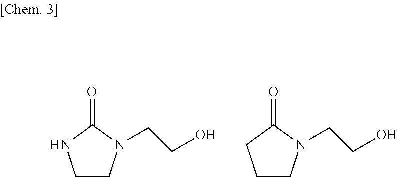 [SMILES: O=C1NCCN1CCO ; O=C1CCCN1CCO]

The component (A) is most preferably 1-(2-hydroxyethyl)-2-imidazolidinone.

The component (A) may be a salt of the cyclic carboxamide derivative represented by Formula (1). The kind of the salt is not particularly limited as long as the salt is pharmacologically acceptable, and the salt may be an inorganic salt or an organic salt. Examples of the inorganic salt include a hydrochloride, a sulfate, a phosphate, a hydrobromide, a sodium salt, a potassium salt, a magnesium salt, a calcium salt, a magnesium salt, and an ammonium salt. Examples of the organic salt include an acetate, a lactate, a maleate, a fumarate, a tartrate, a methanesulfonate, a p-toluenesulfonate, a triethanolamine salt, and an amino acid salt.

One or more kinds of the component (A) can be blended. The amount of the component (A) blended is preferably 1 to 5 mass %, and more preferably 2 to 4 mass % with respect to the total amount of the cosmetic.

### (B) Straight-Chain Higher Fatty Acid Having 16 or More Carbon Atoms

The cosmetic according to the present invention comprises (B) a straight-chain higher fatty acid having 16 or more carbon atoms.

The number of carbon atoms in the component (B) is preferably 18 or more. The upper limit of the number of carbon atoms is preferably 25 or less, more preferably 24 or less, and still more preferably 22 or less.

Examples of the component (B) include saturated fatty acids such as palmitic acid, stearic acid, arachidic acid, behenic acid, and lignoglyceric acid and unsaturated fatty acids such as palmitoleic acid, oleic acid, linoleic acid, α-linolenic acid, γ-linolenic acid, arachidonic acid, eicosapentaenoic acid, and docosahexaenoic acid. The component (B) is preferably selected from the group consisting of behenic acid and stearic acid.

Examples of the neutralizer (for alkali) that forms a soap with the component (B) include hydroxides of alkali metals such as potassium hydroxide and sodium hydroxide, basic nitrogen-containing compounds such as 2-amino-2-methyl-1-propanol, 2-amino-2-methyl-1,3-propanediol, triethanolamine, diethanolamine, monoethanolamine, triisopropanolamine, 2-amino-2-hydroxymethyl-1,3-propanediol, L-arginine, L-lysine, and N-alkyltaurine salts. One or more kinds of these neutralizers can be blended, and the amount of the neutralizer blended is appropriately determined according to the equivalent of the component (B) to be used.

One or more kinds of the component (B) can be blended. The amount of the component (B) blended is preferably 0.1 to 3 mass %, and more preferably 0.3 to 1 mass % with respect to the total amount of the cosmetic.

The cosmetic according to the present invention can include a higher fatty acid (for example, a branched higher fatty acid or a straight-chain higher fatty acid having less than 16 carbon atoms) other than the component (B) as long as an effect of the present invention is not impaired, and the amount of the higher fatty acid blended other than the component (B) is preferably 0.10 mass % or less, more preferably 0.05 mass % or less, still more preferably 0.01 mass % or less, and still even more preferably 0 mass % (that is, not blended) with respect to the total amount of the cosmetic.

### (C) Higher Alcohol

The cosmetic according to the present invention comprises (C) a higher alcohol. The component (C) is preferably a monovalent higher alcohol, and is preferably a saturated or unsaturated monovalent aliphatic alcohol.

The component (C) may be linear or branched, but is preferably linear. The component (C) is preferably a monovalent higher alcohol that is solid at a normal temperature because such a monovalent higher alcohol is likely to form an interfacial film such as a gel film or a liquid crystal film based on a lamellar crystal or the like with the component (B) and the component (D) at a normal temperature or higher. Here, in the present invention, the normal temperature means a range of 5 to 35° C.

Examples of the component (C) include higher alcohols having a melting point of 40° C. or higher such as monovalent aliphatic alcohols having 10 to 30 carbon atoms, and specific examples thereof include stearyl alcohol, isostearyl alcohol, oleyl alcohol, octyldodecanol, cholesterol, sitosterol, cetyl alcohol, cetostearyl alcohol, decyltetradecanol, phytosterol, hexyldecanol, behenyl alcohol, lauryl alcohol, lanolin alcohol, and hydrogenated lanolin alcohol. From the viewpoint of emulsion stability, the component (C) is preferably selected from the group consisting of cetyl alcohol, isostearyl alcohol, stearyl alcohol, and behenyl alcohol.

One or more kinds of the component (C) can be blended. The amount of the component (C) blended is preferably 0.1 to 5 mass %, and more preferably 1 to 4 mass % with respect to the total amount of the cosmetic.

### (D) Nonionic Surfactant

The cosmetic according to the present invention comprises (D) a nonionic surfactant.

The component (D) is not particularly limited, but is preferably selected from the group consisting of polyoxyalkylene glycol fatty acid esters and polyether-modified silicones.

Examples of the polyoxyalkylene fatty acid ester include polyethylene glycol monostearate, polyethylene glycol monooleate, polyethylene glycol monolaurate (hereinafter, abbreviated as PEG) (10), PEG (10) monostearate, PEG (25) monostearate, PEG (40) monostearate, PEG (45) monostearate, PEG (55) monostearate, PEG (100) monostearate, PEG (10) monooleate, PEG distearate, PEG diisostearate, PEG (40) hydrogenated castor oil, PEG (50) hydrogenated castor oil, PEG (60) hydrogenated castor oil, PEG (100) hydrogenated castor oil, hexaglyceryl monolaurate, hexaglyceryl monomyristate, hexaglyceryl monostearate, hexaglyceryl monooleate, decaglyceryl monolaurate, decaglyceryl monomyristate, decaglyceryl monostearate, decaglyceryl monoisostearate, decaglyceryl monooleate, decaglyceryl distearate, decaglyceryl diisostearate, polyoxyethylene (POE) (5) glyceryl monostearate, POE (15) glyceryl monostearate, POE (5) glyceryl monooleate, POE (15) glyceryl monooleate, PEG (8) glyceryl isostearate, PEG (10) glyceryl isostearate, PEG (15) glyceryl isostearate, PEG (20) glyceryl isostearate, PEG (25) glyceryl isostearate, PEG (30) glyceryl isostearate, PEG (40) glyceryl isostearate, PEG (50) glyceryl isostearate, and PEG (60) glyceryl isostearate.

Examples of the polyether-modified silicones include PEG-9 polydimethylsiloxyethyl dimethicone, PEG-10 dimethicone, lauryl PEG-9 polydimethylsiloxyethyl dimethicone, and cetyl PEG/PPG-10/1 dimethicone.

One or more kinds of the component (D) can be blended. The amount of the component (D) blended is preferably 0.1 to 3 mass %, and more preferably 0.3 to 2 mass % with respect to the total amount of the cosmetic.

### (E) Oil

The cosmetic according to the present invention comprises (E) an oil. The component (E) is different from the components (B) and (C). Examples of the component (E) include hydrocarbon oils, silicone oils, ester oils, liquid oils, solid oils, and semi-solid oils, and the component (E) is preferably selected from the group consisting of hydrocarbon oils, silicone oils, ester oils, and solid oils.

Examples of the hydrocarbon oils include isododecane, isohexadecane, isoparaffin, mineral oil (liquid paraffin), ozokerite, squalane, pristane, paraffin, ceresin, squalene, petrolatum, microcrystalline wax, hydrogenated polyisobutene, and hydrogenated polydecene.

Examples of the silicone oils include chain polysiloxanes (such as dimethicone, diphenylsiloxy phenyl trimethicone, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, various modified polysiloxanes (such as an amino-modified polysiloxane, an alkyl-modified polysiloxane, and a fluorine-modified polysiloxane), and acrylic silicones, and chain polysiloxanes are preferable.

Examples of the ester oils include octyl octanoate, nonyl nonanoate, cetyl octanoate, isopropyl myristate, octyldodecyl myristate, isopropyl palmitate, ethylhexyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glycol pivalate, diisostearyl malate, glyceryl di-2-heptylundecanoate, glyceryl diisostearate, trimethylolpropane tri-2-ethylhexanoate, trimethylolpropane triisostearate, pentaerythritol tetra-2-ethylhexanoate, glyceryl tri-2-ethylhexanoate (triethylhexanoin), glyceryl trioctanoate, glyceryl triisopalmitate, trimethylolpropane triisostearate, cetyl 2-ethylhexanoate-2-ethylhexyl palmitate, glyceryl trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamate-2-octyldodecyl ester ((phytosteryl/octyldodecyl) lauroyl glutamate), di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, cetyl ethylhexanoate, and phytosteryl macadamiate.

Examples of the liquid oils include avocado oil, Camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil. Examples of the solid oils include cacao butter, coconut oil, hardened coconut oil, hydrogenated palm oil, palm kernel oil, palm oil, Japan wax kernel oil, hardened oils, Japan wax, and hardened castor oil. Examples of the semi-solid oils include shea butter, partially hydrogenated coconut oil, and partially hydrogenated jojoba oil.

One or more kinds of the component (E) can be blended. The amount of the component (E) blended is preferably 0.5 to 40 mass %, more preferably 5 to 30 mass %, and still more preferably 15 to 25 mass % with respect to the total amount of the cosmetic.

### (F) Water

The cosmetic according to the present invention comprises (F) water. As the water, water used in cosmetics, quasi-drugs, and the like can be used, and for example, purified water, ion-exchanged water, tap water, or the like can be used.

The amount of the water blended is preferably 10 to 80 mass %, more preferably 15 to 60 mass %, and particularly preferably 20 to 45 mass % with respect to the total amount of the cosmetic according to the present invention.

### (G) Alkyl Glyceryl Ether

The cosmetic according to the present invention can further comprise (G) an alkyl glyceryl ether.

The component (D) is a monoether or a diether of an alcohol and glycerin, and is preferably a monoether of an alcohol in which the number of carbon atoms is 14 to 20 and glycerin. Examples of the component (D) include batyl alcohol and chimyl alcohol.

The amount of the component (G) blended is preferably 0.1 to 5 mass %, and more preferably 0.3 to 3 mass % with respect to the total amount of the cosmetic.

### (H) Nicotinic Acid or its Derivative

The cosmetic according to the present invention can further comprise (H) nicotinic acid or its derivative.

Examples of the derivative of nicotinic acid include nicotinic acid amide, benzyl nicotinate, tocopherol nicotinate, and nicotinic acid β-butoxy ester, and among these, nicotinic acid amide is particularly preferable. Here, nicotinic acid amide is also referred to as nicotinamide or niacinamide, is a derivative of nicotinic acid, and is an amide compound of nicotinic acid (vitamin B3/niacin). Nicotinic acid amide is a water-soluble vitamin and a substance known as one of vitamin B complex, and may be extracted from a natural product (rice bran or the like) or can be synthesized with a known method. For example, those listed in the Japanese Pharmacopoeia Eighteenth Edition can be used.

One or more kinds of the component (H) can be blended. The amount of the component (H) blended is preferably 0.1 to 10 mass %, and more preferably 0.5 to 8 mass % with respect to the total amount of the cosmetic.

In the cosmetic according to the present invention, an optional component usually used in cosmetics and pharmaceuticals can be blended in addition to the above-described components. As the optional component, for example, another component usually used in cosmetics, such as a thickener, a moisturizer, a lower alcohol, a metal ion sequestering agent, an antioxidant, a preservative, or a drug, can be blended, and one or more kinds thereof can be blended as long as an effect of the present invention is exhibited.

Examples of the thickener include gum arabic, carrageenan, karaya gum, tragacanth gum, carob gum, quince seed (Pyrus cydonia), casein, dextrin, gelatin, sodium pectinate, sodium alginate, methylcellulose, ethylcellulose, CMC, hydroxyethylcellulose, hydroxypropylcellulose, PVA, PVM, PVP, sodium polyacrylate, a carboxyvinyl polymer (carbomer), a (dimethylacrylamide/sodium acryloyldimethyltaurate) crosspolymer, an (ammonium acryloyldimethyltaurate/VP) copolymer, an (ammonium acryloyldimethyltaurate beheneth-25 methacrylate) crosspolymer, a (sodium acrylate/sodium acryloyldimethyltaurate) copolymer, a (PEG-240/decyltetradeceth-20/HDI) copolymer, locust bean gum, guar gum, tamarind gum, dialkyldimethylammonium sulfate cellulose, xanthan gum, magnesium aluminum silicate, bentonite, hectorite, laponite, and silicic anhydride.

Examples of the moisturizer include polyethylene glycol, propylene glycol, dipropylene glycol (DPG), glycerin, 1,3-butylene glycol (BG), erythritol, xylitol, sorbitol, maltitol, PEG/PPG-14/7 dimethyl ether, chondroitin sulfate, hyaluronic acid, mucoitinsulfuric acid, charonin acid, atelocollagen, cholesteryl-12-hydroxystearate, sodium lactate, bile acid salts, dl-pyrrolidone carboxylates, short-chain soluble collagen, a chestnut rose extract, a yarrow extract, and a melilot extract.

Examples of the lower alcohol include ethanol, 1-propanol, 2-propanol, isobutyl alcohol, and t-butyl alcohol.

Examples of the metal ion sequestering agent include 1-hydroxyethane-1,1-diphosphonic acid, 1-hydroxyethane-1,1-diphosphonic acid tetrasodium salt, edetate disodium (EDTA-2Na), edetate trisodium, edetate tetrasodium, sodium citrate, sodium polyphosphate, sodium metaphosphate, gluconic acid, phosphoric acid, citric acid, succinic acid, edetic acid, and trisodium hydroxyethyl ethylenediamine triacetate.

Examples of the antioxidant include dibutylhydroxytoluene, butylhydroxyanisole, sodium pyrosulfite, and gallic acid esters.

Examples of the preservative include paraoxybenzoic acid esters such as methylparaben, ethylparaben, and butylparaben, benzoic acid, salicylic acid, sorbic acid, parachlorometacresol, hexachlorophene, benzalkonium chloride, chlorhexidine chloride, trichlorocarbanilide, photosensitizers, and phenoxyethanol.

Examples of the drug include ascorbic acid (vitamin C), tranexamic acid, kojic acid, ellagic acid, arbutin, alkoxysalicylic acid, glycyrrhizic acid, tocopherol, retinol, salts and derivatives thereof (for example, sodium L-ascorbate, L-ascorbic acid ester magnesium salt, L-ascorbic acid glucoside, 2-O-ethyl-L-ascorbic acid, 3-O-ethyl-L-ascorbic acid, 4-methoxysalicylic acid sodium salt, 4-methoxysalicylic acid potassium salt, dipotassium glycyrrhizinate, stearyl glycyrrhizinate, tocopherol acetate, retinol acetate, and retinol palmitate), caffeine, tannin, verapamil and its derivatives, a licorice extract, glabridin, a hot water extract of Pyracantha fortuneana seed, various herbal medicines, hydrolyzed silk, hydrolyzed conchiolin, a tea extract, a Potentilla erecta root extract, an Angelica keiskei leaf/stem extract, an Aloe barbadensis leaf extract, a cherry leaf extract, an Angelica acutiloba Kitagawa root extract, a Citrus depressa peel extract, an Iris florentina root extract, an Eucheuma serra/Grateloupia sparsa/Saccharina angustata/Ulva linza/Undaria pinnatifida extract, a Typha angustifolia spike extract, an Isodonis japonicus leaf/stalk extract, a Camellia japonica seed extract, a Saccharina angustata/Undaria pinnatifida extract, a Bupleurum falcatum root extract, a Nasturtium officinale leaf/stem extract, a Cinnamomum cassia bark extract, a rosemary leaf oil, a lavender oil, glutamic acid, trimethylglycine, chlorphenesin, and menthoxypropanediol.

In addition, an ultraviolet absorber, a powder, a colorant, an aroma chemical, and the like can also be appropriately blended.

The method for producing the cosmetic according to the present invention is not particularly limited, but the cosmetic can be prepared with a known method such as a dispersion method or an aggregation method. The dispersion method is a method of finely dividing a mass in a dispersion layer by a mechanical force. The aggregation method is a colloid preparation method in which an interfacial chemical characteristic is used, and is a method of changing a uniformly dissolved state to a supersaturated state by some means to generate a dispersion layer.

The cosmetic according to the present invention is advantageously prepared with a liquid crystal emulsification method among aggregation methods. In the vicinity of the interface of emulsified particles prepared with the liquid crystal emulsification method, an interfacial film such as a gel film or a liquid crystal film based on lamellar liquid crystal or the like is easily formed. As a result, aggregation and coalescence of emulsified particles are suppressed, and the emulsion stability is improved.

The cosmetic according to the present invention can be produced, for example, with the following method.

(B) A straight-chain higher fatty acid having 16 or more carbon atoms, (C) a higher alcohol, (D) a nonionic surfactant, (E) an oil, and optionally an oil-soluble optional component are mixed while being heated to about 70 to 90° C. to prepare an oil phase solution. Next, (A) a cyclic carboxamide derivative or its salt and optionally a water-soluble optional component are mixed with (F) water while being heated to about 70 to 90° C. to prepare an aqueous phase solution. To this aqueous phase solution being stirred, the oil phase solution is gradually added, then the resulting solution is cooled to a normal temperature, and thus an oil-in-water cosmetic can be prepared.

In the conventional liquid crystal emulsification method, it has been confirmed that an interfacial film such as a gel film or a liquid crystal film based on lamellar liquid crystal or the like is formed around emulsified particles at the stage of cooling to a normal temperature, and therefore it is considered that such an interfacial film is also formed around the emulsified particles of the present disclosure at the similar stage.

Examples of the cosmetic according to the present invention include skin care cosmetics (such as lotions, milky lotions, creams, beauty essences, packs, and masks), makeup cosmetics (such as foundations and makeup bases), skin cleaning agents (such as face washes and makeup removers), sunscreen cosmetics, and ointments. These embodiments are merely examples, and the cosmetic according to the present invention is not limited to these embodiments.

### EXAMPLES

The present invention will be described specifically with reference to the following Examples, but the present invention is not limited to these Examples. The content is shown in mass % with respect to the total amount unless otherwise specified.

### Examples 1 to 12 and Comparative Examples 1 to 3

Oil-in-water emulsion cosmetics of Examples 1 to 3 and Comparative Examples 1 to 4 were prepared with a liquid crystal emulsification method described below at combinations shown in Table 1. The numerical values in the table are shown in mass %.

While heating was performed at 70 to 90° C., (E) an oil was uniformly mixed, and then (B) a straight-chain higher fatty acid having 16 or more carbon atoms, (C) a higher alcohol, and (D) a nonionic surfactant were further mixed to prepare an oil phase solution. Subsequently, other components were added to (F) water while heating was performed at 70 to 90° C., and mixed uniformly to prepare an aqueous phase solution. While the aqueous phase solution was heated to 70 to 90° C., the oil phase solution was gradually added and uniformly dispersed with a dispenser, and then the resulting solution was cooled to a normal temperature to obtain an oil-in-water emulsion cosmetic.

TABLE 1

| | | Example | | | Comparative Example | | | |
|---|---|---|---|---|---|---|---|---|
| | | 1 | 2 | 3 | 1 | 2 | 3 | 4 |
| 1-(2-Hydroxyethyl)-2-imidazolidinone | | 1.5 | 3 | 1.5 | 0 | 1.5 | 1.5 | 1.5 |
| Behenic acid | | 0.6 | 0.6 | 0.6 | 0.6 | 0 | 0.6 | 0.6 |
| Behenyl alcohol | | 2.5 | 2.5 | 2.5 | 2.5 | 2.5 | 2.5 | 0 |
| PEG-60 glyceryl isostearate | | 0.5 | 0.5 | 0.5 | 0.5 | 0.5 | 0 | 0.5 |
| PEG-10 dimethicone | | 0.5 | 0.5 | 0.5 | 0.5 | 0.5 | 0 | 0.5 |
| Hydrogenated polyisobutene | | 1 | 1 | 1 | 1 | 1 | 1 | 1 |
| Triethylhexanoin | | 3 | 3 | 3 | 3 | 3 | 3 | 3 |
| (Phytosteryl/octyldodecyl) lauroyl glutamate | | 2 | 2 | 2 | 2 | 2 | 2 | 2 |
| Diphenylsiloxy phenyl trimethicone | | 5 | 5 | 5 | 5 | 5 | 5 | 5 |
| Dimethicone | | 3 | 3 | 3 | 3 | 3 | 3 | 3 |
| Hydrogenated palm oil | | 0.5 | 0.5 | 0.5 | 0.5 | 0.5 | 0.5 | 0.5 |
| Palm kernel oil | | 0.3 | 0.3 | 0.3 | 0.3 | 0.3 | 0.3 | 0.3 |
| Palm oil | | 0.3 | 0.3 | 0.3 | 0.3 | 0.3 | 0.3 | 0.3 |
| Water | | Balance | Balance | Balance | Balance | Balance | Balance | Balance |
| Batyl alcohol | | 1 | 1 | 1 | 1 | 1 | 1 | 0 |
| Nicotinic acid amide | | 0 | 0 | 5 | 0 | 0 | 0 | 0 |
| Potassium hydroxide | | 0.1 | 0.1 | 0.1 | 0.1 | 0 | 0.1 | 0.1 |
| (Dimethylacrylamide/sodium | | 0.5 | 0.5 | 0.5 | 0.5 | 0.5 | 0.5 | 0.5 |
| acryloyldimethyltaurine) crosspolymer | | | | | | | | |
| (PEG-240/decyltetradeceth-20/HDI) copolymer | | 0.1 | 0.1 | 0.1 | 0.1 | 0.1 | 0.1 | 0.1 |
| Xanthan gum | | 0.05 | 0.05 | 0.05 | 0.05 | 0.05 | 0.05 | 0.05 |
| Glycerin | | 15 | 15 | 15 | 15 | 15 | 15 | 15 |
| DPG | | 10 | 10 | 10 | 10 | 10 | 10 | 10 |
| BG | | 10 | 10 | 10 | 10 | 10 | 10 | 10 |
| PEG/PPG-14/7 dimethyl ether | | 1 | 1 | 1 | 1 | 1 | 1 | 1 |
| Ethanol | | 3 | 3 | 3 | 3 | 3 | 3 | 3 |
| EDTA-2Na | | 0.03 | 0.03 | 0.03 | 0.03 | 0.03 | 0.03 | 0.03 |
| Citric acid | | 0.1 | 0.1 | 0.1 | 0.1 | 0.1 | 0.1 | 0.1 |
| Sodium metaphosphate | | 0.1 | 0.1 | 0.1 | 0.1 | 0.1 | 0.1 | 0.1 |
| Sodium pyrosulfite | | 0.01 | 0.01 | 0.01 | 0.01 | 0.01 | 0.01 | 0.01 |
| Phenoxyethanol | | 0.5 | 0.5 | 0.5 | 0.5 | 0.5 | 0.5 | 0.5 |
| Tocopherol | | 0.1 | 0.1 | 0.1 | 0.1 | 0.1 | 0.1 | 0.1 |
| Bupleurum falcatum root extract | | 0.1 | 0.1 | 0.1 | 0.1 | 0.1 | 0.1 | 0.1 |
| Cinnamomum cassia bark extract | | 0.1 | 0.1 | 0.1 | 0.1 | 0.1 | 0.1 | 0.1 |
| Typha angustifolia spike extract | | 0.1 | 0.1 | 0.1 | 0.1 | 0.1 | 0.1 | 0.1 |
| Isodonis japonicus leaf/stalk extract | | 0.1 | 0.1 | 0.1 | 0.1 | 0.1 | 0.1 | 0.1 |
| 2-O-ethyl ascorbic acid | | 0.1 | 0.1 | 0.1 | 0.1 | 0.1 | 0.1 | 0.1 |
| Hydrolyzed silk | | 0.1 | 0.1 | 0.1 | 0.1 | 0.1 | 0.1 | 0.1 |
| Camellia japonica seed extract | | 0.1 | 0.1 | 0.1 | 0.1 | 0.1 | 0.1 | 0.1 |
| Angelica acutiloba Kitagawa root extract | | 0.1 | 0.1 | 0.1 | 0.1 | 0.1 | 0.1 | 0.1 |
| Cherry leaf extract | | 0.1 | 0.1 | 0.1 | 0.1 | 0.1 | 0.1 | 0.1 |
| Tea extract | | 0.1 | 0.1 | 0.1 | 0.1 | 0.1 | 0.1 | 0.1 |
| Hydrolyzed conchiolin | | 0.1 | 0.1 | 0.1 | 0.1 | 0.1 | 0.1 | 0.1 |
| Citrus depressa peel extract | | 0.1 | 0.1 | 0.1 | 0.1 | 0.1 | 0.1 | 0.1 |
| Iris florentina root extract | | 0.1 | 0.1 | 0.1 | 0.1 | 0.1 | 0.1 | 0.1 |
| Eucheuma serra/Grateloupia sparsa/Saccharina | | 0.05 | 0.05 | 0.05 | 0.05 | 0.05 | 0.05 | 0.05 |
| angustata/Ulva linza/Undaria pinnatifida extract | | | | | | | | |
| Saccharina angustata/Undaria pinnatifida extract | | 0.05 | 0.05 | 0.05 | 0.05 | 0.05 | 0.05 | 0.05 |
| Nasturtium officinale leaf/stem extract | | 0.1 | 0.1 | 0.1 | 0.1 | 0.1 | 0.1 | 0.1 |
| Aluminium hydroxide | | 0.2 | 0.2 | 0.2 | 0.2 | 0.2 | 0.2 | 0.2 |
| Mica | | 1.5 | 1.5 | 1.5 | 1.5 | 1.5 | 1.5 | 1.5 |
| Titanium oxide | | 1.5 | 1.5 | 1.5 | 1.5 | 1.5 | 1.5 | 1.5 |
| Silica | | 0.5 | 0.5 | 0.5 | 0.5 | 0.5 | 0.5 | 0.5 |
| Iron oxide | | 0.01 | 0.01 | 0.01 | 0.01 | 0.01 | 0.01 | 0.01 |
| Aroma chemical | | 0.2 | 0.2 | 0.2 | 0.2 | 0.2 | 0.2 | 0.2 |
| Total | | 100 | 100 | 100 | 100 | 100 | 100 | 100 |
| Stability evaluation | | ∘ | ∘ | ∘ | ∘ | x | x | x |
| pH | | 6.89 | 7.06 | 6.92 | 6.95 | — | — | — |
| Viscosity (#6, 10 rpm, 30° C.) mPa · s | | 20100 | 22200 | 19700 | 20700 | — | — | — |
| Average emulsified particle size μm | | 2.5 | 2.5 | 2.5 | 2.5 | — | — | — |
| Usability | Absence of sticky feel | AA | AA | AA | AA | — | — | — |
| evaluation | Moist feel | A | A | A | C | — | — | — |
| | Smooth feel | B | A | B | C | — | — | — |
| | Mellow feel | A | AA | AA | C | — | — | — |

### [Stability Evaluation]

The cosmetic prepared above was observed visually and with an optical microscope immediately after preparation, and the stability was evaluated in accordance with the following criteria. The obtained results are as shown in Table 1.
◯: Separation of the oil was not visually confirmed, and remarkable coalescence and enlargement of the emulsified particles were not confirmed with an optical microscope.
x: Separation of the oil was visually confirmed, and remarkable coalescence and enlargement of the emulsified particles were confirmed with an optical microscope.

### [Viscosity]

The cosmetics of Examples 1 to 3 and Comparative Example 1 prepared above were measured at 30° C. using a BH type viscometer (No. 6 rotor, rotation speed 10 rpm). The obtained results are as shown in Table 1.

### [Average Emulsified Particle Size]

The cosmetics of Examples 1 to 3 and Comparative Example 1 immediately after preparation described above were observed with a microscope, the diameters of 100 emulsified particles were measured, and the average of the diameters was regarded as the average particle size. The obtained results are as shown in Table 1.

### [Usability Evaluation]

The cosmetic prepared above was applied to the faces of 10 expert panelists, and the panelists evaluated “absence of a sticky feel” of the skin after the application, the “moist feel” of the skin after the application, the “smooth feel” of the skin after the application, and the “mellow feel” of the base. Determination was made in accordance with the following criteria on the basis of the evaluation of each expert panelist. The obtained results are as shown in Table 1.

### “Absence of Sticky Feel”

AA: Nine or more of the 10 panelists answered that a sticky feel was absent.
A: Seven or more and 8 or less of the 10 panelists answered that a sticky feel was absent.
B: Five or more and 6 or less of the 10 panelists answered that a sticky feel was absent.
C: Three or more and 4 or less of the 10 panelists answered that a sticky feel was absent.
D: One or more and 2 or less of the 10 panelists answered that a sticky feel was absent.
E: All of the 10 panelists answered that a sticky feel was present.

### “Moist Feel”

AA: Nine or more of the 10 panelists answered that a moist feel of the skin was present.
A: Seven or more and 8 or less of the 10 panelists answered that a moist feel of the skin was present.
B: Five or more and 6 or less of the 10 panelists answered that a moist feel of the skin was present.
C: Three or more and 4 or less of the 10 panelists answered that a moist feel of the skin was present.
D: One or more and 2 or less of the 10 panelists answered that a moist feel of the skin was present.
E: All of the 10 panelists answered that a moist feel of the skin was absent.

### “Smooth Feel”

AA: Nine or more of the 10 panelists answered that a smooth feel of the skin was present.
A: Seven or more and 8 or less of the 10 panelists answered that a smooth feel of the skin was present.
B: Five or more and 6 or less of the 10 panelists answered that a smooth feel of the skin was present.
C: Three or more and 4 or less of the 10 panelists answered that a smooth feel of the skin was present.
D: One or more and 2 or less of the 10 panelists answered that a smooth feel of the skin was present.
E: All of the 10 panelists answered that a smooth feel of the skin was absent.

### “Mellow Feel”

AA: Nine or more of the 10 panelists answered that a mellow feel was present.
A: Seven or more and 8 or less of the 10 panelists answered that a mellow feel was present.
B: Five or more and 6 or less of the 10 panelists answered that a mellow feel was present.
C: Three or more and 4 or less of the 10 panelists answered that a mellow feel was present.
D: One or more and 2 or less of the 10 panelists answered that a mellow feel was present.
E: All of the 10 panelists answered that a mellow feel was absent.

1. An oil-in-water emulsion cosmetic comprising:
(A) a cyclic carboxamide derivative or a salt of the cyclic carboxamide derivative, the cyclic carboxamide derivative represented by Formula (1): 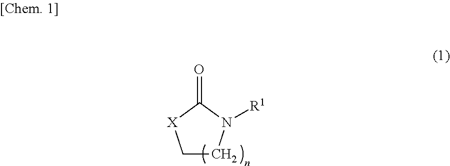
wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) a straight-chain higher fatty acid having 16 or more carbon atoms;
(C) a higher alcohol;
(D) a nonionic surfactant;
(E) an oil; and
(F) water.

2. The cosmetic according to claim 1, wherein
in Formula (1) of the component (A),
R¹ represents a hydroxyalkyl group having 1 to 3 carbon atoms,
X represents —CH₂— or —NH—, and
n is 1.

3. The cosmetic according to claim 1, wherein the component (A) is 1-(2-hydroxyethyl)-2-imidazolidinone.

4. The cosmetic according to claim 1, wherein an amount of the component (A) blended is 1 to 5 mass % with respect to a total amount of the cosmetic.

5. The cosmetic according to claim 1, wherein the component (B) is selected from the group consisting of behenic acid and stearic acid.

6. The cosmetic according to claim 1, wherein an amount of the component (B) blended is 0.1 to 3 mass % with respect to the total amount of the cosmetic.

7. The cosmetic according to claim 1, wherein the component (C) is selected from the group consisting of cetyl alcohol, isostearyl alcohol, stearyl alcohol, and behenyl alcohol.

8. The cosmetic according to claim 1, wherein an amount of the component (C) blended is 0.1 to 5 mass % with respect to the total amount of the cosmetic.

9. The cosmetic according to claim 1, wherein the component (D) is selected from the group consisting of polyoxyalkylene glycol fatty acid esters and polyether-modified silicones.

10. The cosmetic according to claim 1, further comprising (G) an alkyl glyceryl ether.

11. The cosmetic according to claim 1, further comprising (H) nicotinic acid or a derivative of nicotinic acid.
